(19) United States
(12) Patent Application Publication
(10) Pub. No.: US 2019/0307487 A1
(43) Pub. Date: Oct. 10, 2019

(54) PEEL-AWAY ELECTRODE PLACEMENT CATHETER

(71) Applicant: Brown University, Providence, RI (US)

(72) Inventors: Wael Asaad, Westwood, MA (US); Shane Lee, Providence, RI (US); Owen Leary, Providence, RI (US); Noah Trac, Covina, CA (US); Zakir Tahiry, Providence, RI (US); Rohan Rastogi, Westford, MA (US)

(21) Appl. No.: 16/277,667

(22) Filed: Feb. 15, 2019

Related U.S. Application Data
(60) Provisional application No. 62/631,256, filed on Feb. 15, 2018.
(60) Provisional application No. 62/657,454, filed on Apr. 13, 2018.

(51) Int. Cl. A61B 17/34; A61N 1/05; A61B 90/11; A61N 1/36

(52) U.S. Cl. CPC A61B 17/3468; A61N 1/0534; A61B 2090/103; A61N 1/3605; A61B 90/11

(57) ABSTRACT

A peel-away electrode placement catheter includes a silicone tube, the silicone tube including perforations longitudinally positioned to spilt the silicone tube in half, a pair of separate metal semi-cylinders positioned within the silicone tube, and a deep brain stimulation (DBS) implantable electrode positioned between the pair of separate metal semi-cylinders.

### CROSS REFERENCE TO RELATED APPLICATIONS

This application claims benefit from U.S. Provisional Patent Application Ser. No. 62/631,256, filed Feb. 15, 2018, and U.S. Provisional Patent Application Ser. No. 62/657,454 filed Apr. 13, 2018, which each are incorporated by reference in their entireties.

### STATEMENT REGARDING GOVERNMENT INTEREST

None.

### BACKGROUND OF THE INVENTION

This invention generally relates to catheters, and more specifically to a peel-away electrode placement catheter.

Deep brain stimulation (DBS) of specific areas of the brain has been used with great success as a clinical treatment for a range of neurological and psychiatric disorders rooted in often Progressive abnormalities of the underlying neural circuitry, including essential tremor, schizophrenia, and Parkinson's disease. More recently DBS has been attempted with some success to treat an even wider range of neuro disorders, including epilepsy, obsessive compulsive disorder (OCD), and major depression. This treatment is increasingly common.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a catheter including a silicone tube, the silicone tube including perforations longitudinally positioned to split the silicone tube in half, and a pair of separate metal semi-cylinders positioned within the silicone tube.

In another aspect, the invention features a peel-away electrode placement catheter including a silicone tube, the silicone tube including perforations longitudinally positioned to split the silicone tube in half, a pair of separate metal semi-cylinders positioned within the silicone tube, and a deep brain stimulation (DBS) implantable electrode positioned between the pair of separate metal semi-cylinders.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1A, FIG. 1B, FIG. 1C and FIG. 1D illustrate an exemplary prior art design of a guide tube.

FIG. 2 illustrates a standard prior art guide tube.

FIG. 3A, FIG. 3B, FIG. 3C, FIG. 3D and FIG. 3E illustrate an exemplary perforated peel-away catheter.

FIG. 4 illustrates exemplary design specifications of the perforated peel-away catheter.

FIG. 5A and FIG. 5B illustrate different perforated peel-away catheter applications.

FIG. 6A and FIG. 6B illustrate additional perforated peel-away catheter applications.

FIG. 7 illustrates an exemplary annulus.

FIG. 8A and FIG. 8B illustrate another exemplary annulus.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The standard electrode insertion catheter (herein referred to as a “guide tube”) that is used to guide the implantation of DBS electrodes is a uniform, inflexible, Hollow cylinder with approximate dimensions of 1.27 millimeter inner diameter, 1.8 mm outer diameter and 21 cm length. Flexible, delicate stimulating electrodes are guided to their deep brain targets through these thin steel guide tubes. Due to their rigid geometry, the guide tubes must then be withdrawn over the extracranially-protruding (“proximal”) end of the implanted DBS electrode after the electrode has been inserted. Because the guide tube must be fully withdrawn after electrode implantation, the connection that must be established between the proximal end of the electrode and the other components of the stimulator system must therefore be established after the guide tube is withdrawn. The procedural step of withdrawing a rigid guide tube there for limits the complexity of the electrical connections that can be established at the proximal end of the electrode, due to the lack of available connector technologies that can be swiftly assembled intraoperatively and also accommodate increased number of electrical channels embedded in the electrode. Connector setups that take significantly more time to assemble intraoperatively are not acceptable to surgeons, even for the benefit of accommodating more electrical channels, because of the increased risk of operative complication with increased surgical time. Further, this constraint limits potential improvements to the therapeutic efficiency of the procedure that might be possible with more intricate stimulating and recording electrodes that require a more complex and time-consuming connector setup (e.g., closed loop DBS). there is a clinical need to improve the surgical procedure of DBS, or the equipment used there in, to maximize the therapeutic efficiency of DBS by enhancing intraoperative testing and usage of such more powerful and complex electrode-connector systems. One goal of the present invention is to provide a guide tube that can be peeled away by the surgeon as it is withdrawn over the electrode.

By obviating the need for the guide tube to be pulled over the proximal end of the electrode the present invention alleviates the limitations on electrode complexity imposed by the fixed guide tube geometry of current devices. A peel-away guide tube opens the possibility of manufacturing more complex electrode connector systems that accommodates a greater number of recording and stimulating channels within the electrode proper, since the peel away tube enables intraoperative implementation of such systems without need for additional assembly during the surgical procedure. The design of the present invention is a longitudinally perforated peel away guide tube with the same external dimensions as the currently standard steel device. It includes deformable silicone rubber with steel semicircular inserts embedded in the silicon between the perforations. Pull tabs are featured to aid intraoperatively splitting the silicone rubber tubing along the perforations as it is withdrawn from the cranium.

The design of the present invention reimagines the uniform steel insertion catheter design typically used in DBS surgery during the electrode insertion step. Specifically, the present design includes (1) an insertion catheter for electrodes, or for other biomedical devices applied or implanted intracranially (such as neurochemical Delivery Systems or measurement systems) whose outer casing is made of silicone, (2) and insertion catheter for electrodes, or for other biomedical devices applied or implanted intracranially, that can split in half longitudinally along predefined preparations such that controlled deformation upon withdrawal from the brain tissue can be achieved, (3) an insertion catheter for electrodes, or for other biomedical devices applied or implanted intracranially, made of an outer silicone casing that also includes dual steel semi-cylinders embedded such that the tube retains its rigidity upon insertion into the brain tissue and until intentional tearing stresses are applied to break it along the perforations, (4) an Insertion catheter for electrodes, or for other biomedical devices applied or implanted intracranially, that is readily deformable with less than 5 N bilateral force applied to opposite sides of the tube, (5) and insertion catheter that need not interrupt pre-established connections between the stimulating or recording electrode implanted through the catheter and the rest of the DBS system, or more broadly the connections between any biomedical device inserted through the catheter and its corresponding extra cranial components.

Using the design of the present invention, the ergonomics of the DBS procedure are not altered, nor additional burden is imposed on the patient or a user neurosurgeon, and all geometric and functional requirements defined by current electrode insertion catheters are met. The present design uniquely leverages and combines material properties of silicon and steel to produce the desired functional outcome. the design effectively opens a gateway to further advances in the therapeutic impact of deep brain stimulation by enabling the use of more complex, versatile stimulating and recording electrodes, towards eventual application of closed-loop DBS.

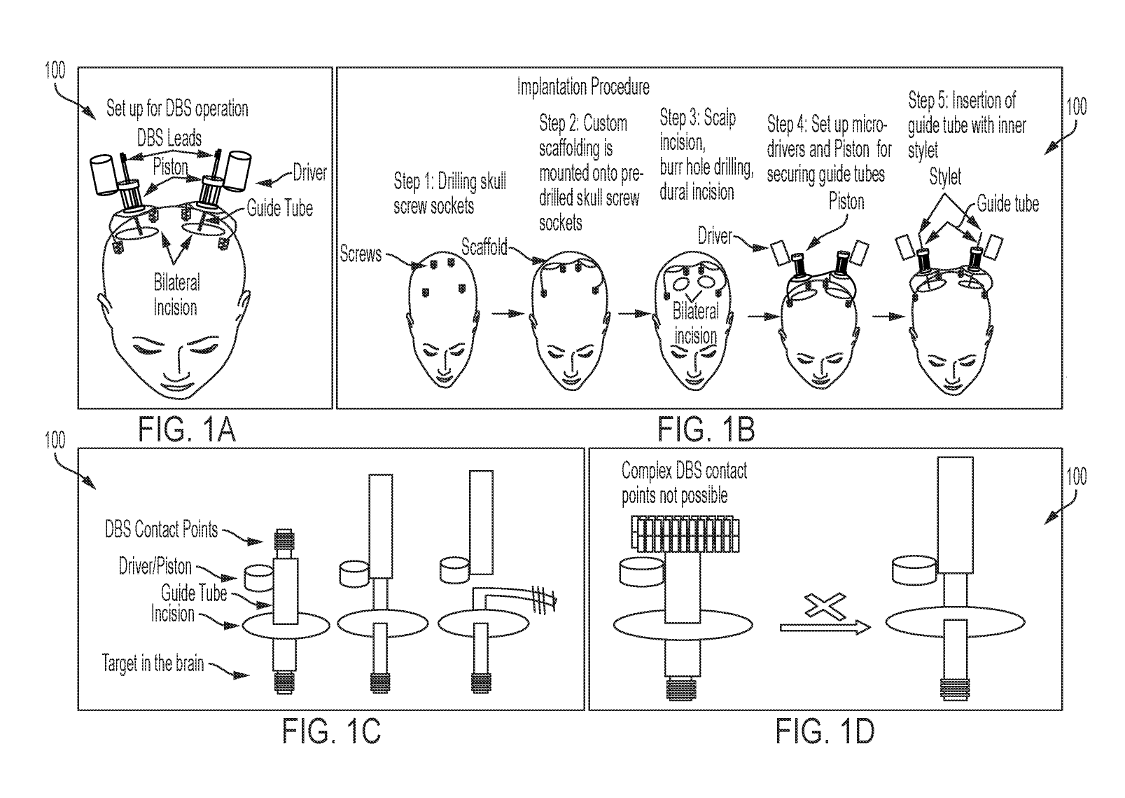

FIG. 1A, FIG. 1B, FIG. 1C and FIG. 1D illustrate an exemplary prior art design of a guide tube 100. More specifically, FIG. 1A illustrates an exemplary DBS procedural setup, FIG. 1B illustrates the exemplary steps of surgical electrode implantation, FIG. 1C illustrates exemplary steps of guide tube removal and FIG. 1D illustrates the clinical problem associated with this device and technique, i.e., an inability to withdraw the guide tube over the distal end.

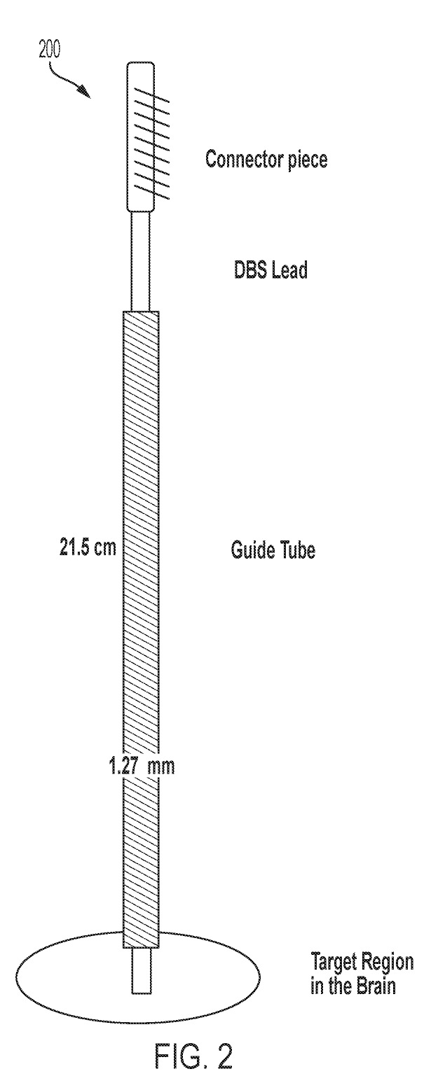

In FIG. 2, a standard prior art guide tube 200 is shown and includes a hollow steel cylinder, with an inner diameter of 1.27 mm, outer diameter of 1.8 mm and a length of 21 cm. Given the rigidity of the guide tube 200, it must be withdrawn over the proximal end of the DBS lead during removal, forcing the neurosurgeon to establish the electrical connections after removal, and limiting the power and versatility of the electrical system used. A primary aim of the present invention used to develop a guide tube that alleviate such limitations, enabling the DBS lead to be manufactured with pre-established connections. Another objective is to design a guide with geometric dimensions that closely adhere to those of the currently used guide tube so as not to interfere with the current ergonomics of the insertion procedure or the size of the burr hole to be drilled in the patient's skull.

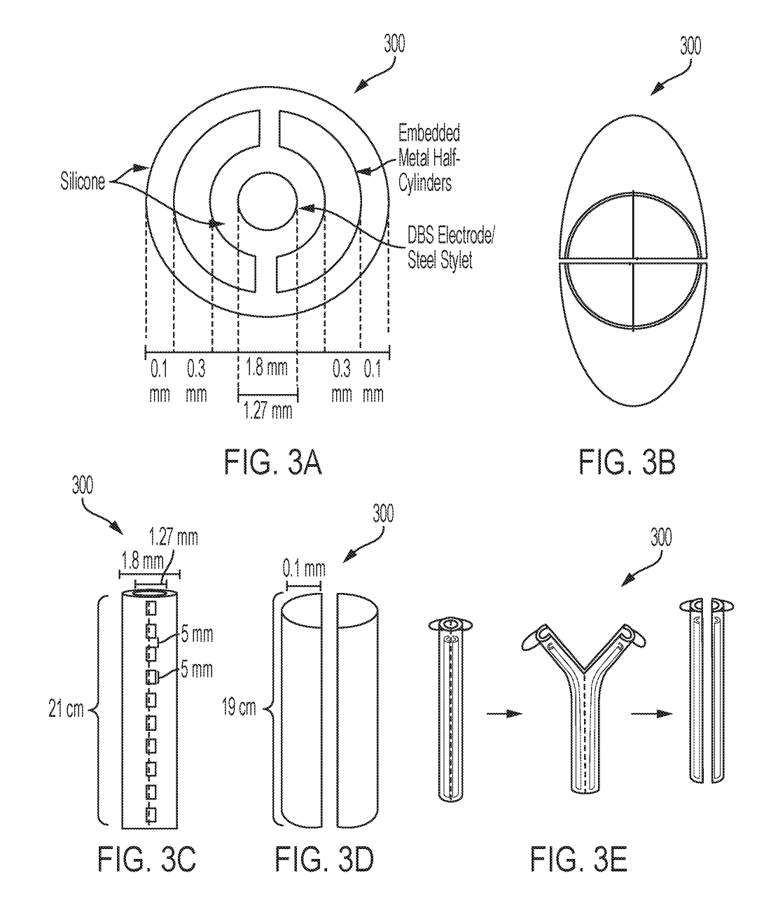

As shown in FIG. 3A, FIG. 3B, FIG. 3C, FIG. 3D and FIG. 3E, an exemplary perforated peel-away guide tube 300 includes deformable silicone rubber with steel semicylinder inserts embedded in the silicon between the perforations. the perforated peel-away guide tube 300 features approximately the same geometry and dimensions as the prior art steel guide tube. Pull tabs are added to aid in intraoperative deformation of the silicone rubber tubing.

More specifically, FIG. 3A illustrates a cross sectional perspective of the silicon and steel guide tube, FIG. 3B illustrates the pull flaps, FIG. 3C illustrates a side view of the silicone tube perforations, FIG. 3D illustrates a side view of the dual steel semi-cylinders, and FIG. 3E illustrates the combined silicone and steel components.

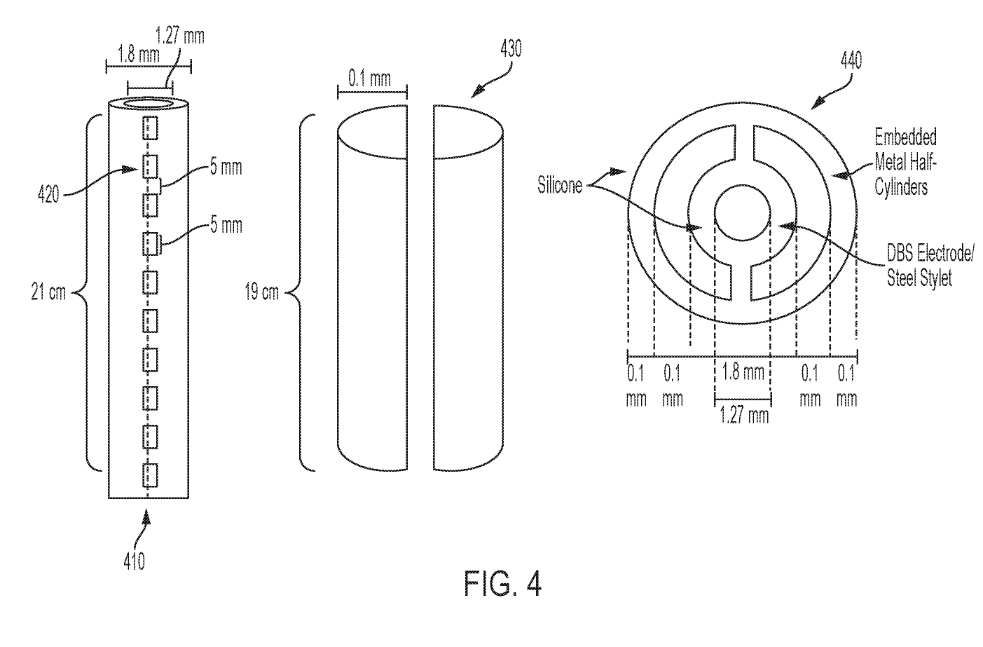

In FIG. 4, exemplary design specifications of the perforated peel-away guide tube 300 are shown. The silicone tube 410 includes an inner diameter of about 1.27 mm, an outer diameter of about 1.8 mm and an over length of about 21 cm. Perforations 420 are spaced equally 5 mm apart. The metal semi-cylinder 430 is about 19 cm in length. In the top view 440, exemplary dimensions of the silicone, embedded metal semi-cylinders and DBS electrode/steel stylet are shown.

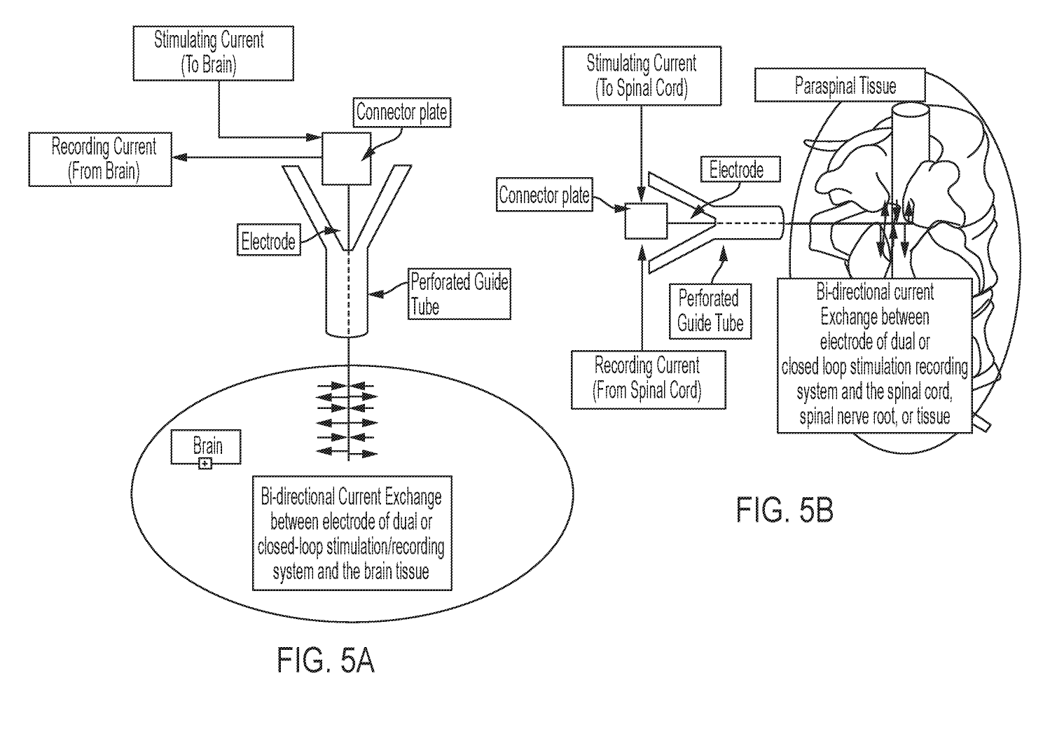

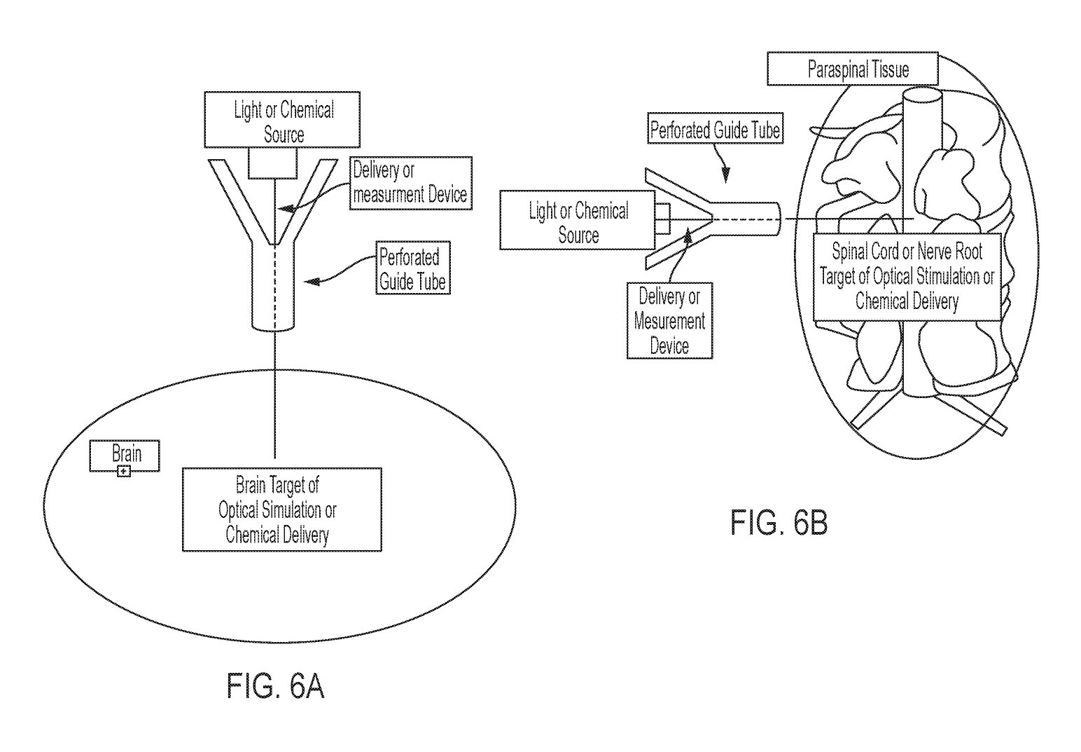

The perforated peel-away guide tube 300 may additionally be implanted intraspinally, and the potential usages for either brain or spine include, but are not limited to, implantation of stimulating electrodes, recording electrodes, electrodes that operate to both record and stimulate simultaneously or distinctly (see FIG. 5A and FIG. 5B), optical recording and stimulation (e.g., optical coherence tomography systems, OCT), and chemical assay and delivery systems (e.g. intrathecal pain medication, neurotransmitter delivery systems, amperometry or voltometry systems) (see FIG. 6A and FIG. 6B). For all of these potential usages, our catheter can be used for devices intended to be implanted either permanently or temporarily. In spinal applications, the device may enable interaction with either the spinal cord proper, or with any of the spinal nerve roots, rootlets, or cauda equina. Furthermore, the device's technical dimensions and specifications as described elsewhere in these application documents may be optimized for use in any of these applications, with potential modifications including but not limited to alterations in internal or external diameter, wall thickness, spacing of metal hemi-cylindrical inserts within the flexible wall, specific grade of materials used for metal or silicone components, and/or including either a closed or open end to the catheter.

Importantly, the perforated peel-away guide tube 300 may be used for many applications not traditionally defined as, nor necessarily related to, DBS, though DBS is one particularly impactful application for which it might be used therapeutically in the short term.

In other embodiments, the perforated peel-away guide tube 300 may include, as a connected or separate component, a cylindrical annulus made of any biocompatible material, including certain plastics or metals, with one or multiple bores each no less than the size of the peel away catheter, with one or multiple hinges that open around the bores, and with eyelets for affixing the cylindrical annulus to the skull. Biocompatible skull screws can be separate or pre-attached and securely but freely spinning in the eyelets. One purpose of the annulus is to discourage peeling of the catheter beneath the cylinder, and to enable one or multiple catheters to be inserted and safely removed above the annulus without inflicting damage on cortical or subcortical tissues.

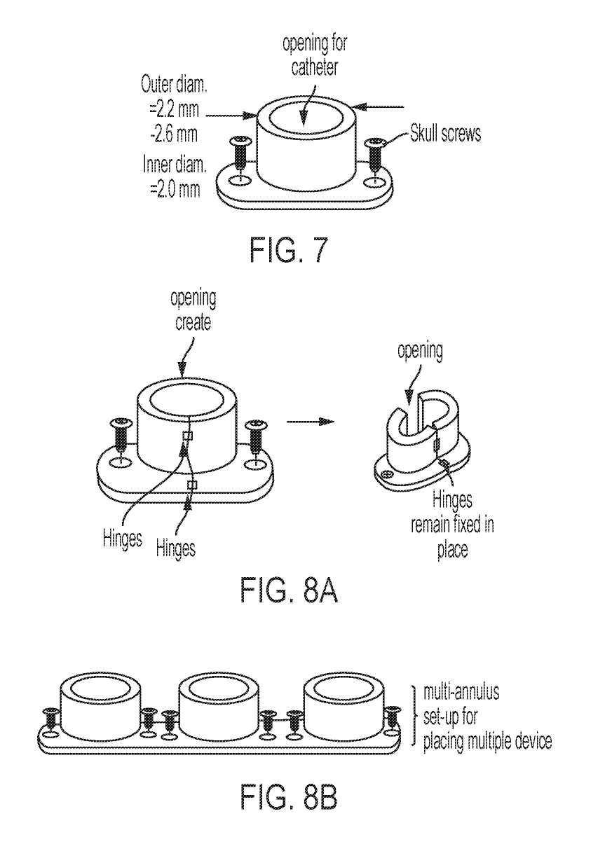

In FIG. 7, an exemplary annulus is constructed of biocompatible material, such as steel with one bore to accommodate passage of a 1.8 mm peel-away catheter and peripheral holes (x2) for fixation to the intraoperatively exposed skull via biocompatible skull screws. A base plate features holes to accommodate the skull screws.

In FIG. 8A and FIG. 8B, the cylindrical annulus design may be modified to accommodate new constraints imposed by a peel-away catheter and the application involved. In FIG. 8A, hinges are implemented to enable easier placement and removal of the annulus after the catheter itself has already been placed, such that the annulus can provide additional as the catheter is peeled. In FIG. 8B, as many trajectories are tested before one is selected for permanent DBS implant via separate electrodes, a multi-annulus array is designed to enable this. Moreover, the base plate may be curved to accommodate anatomical curvature of the cranium.

The foregoing description of the preferred embodiments of the invention is by way of example only, and other variations of the above-described embodiments and methods are provided by the present invention. The embodiments described herein have been presented for purposes of illustration and are not intended to be exhaustive or limiting. Many variations and modifications are possible in light of the foregoing teaching. The invention is limited only by the following claims.

What is claimed is:

1. A catheter comprising:
a silicone tube, the silicone tube including perforations longitudinally positioned to spilt the silicone tube in half; and
a pair of separate metal semi-cylinders positioned within the silicone tube.

2. The catheter of claim 1 further comprising a stimulating or recording electrode positioned between the pair of separate metal semi-cylinders.

3. The catheter of claim 2 further comprising:
a lead linked to the stimulating or recording electrode which contact brain tissue; and
a connector piece linked to the lead.

4. The catheter of claim 1 wherein the silicone tube comprises:
a thickness of 0.1 millimeters;
a length of 21 centimeters; and
an overall diameter of 1.8 millimeters.

5. The catheter of claim 1 wherein each of the separate metal semi-cylinders comprises:
a length of 19 centimeter; and
a radius of 0.3 millimeters.

6. The catheter of claim 2 further comprising a cylindrical annulus set-up constructed of biocompatible material.

7. The catheter of claim 6 further comprising:
the annulus set-up with one or multiple bores each no less than a size of the silicone tube, with one or multiple hinges that open around the bores, and with eyelets for affixing the cylindrical annulus to a human skull.

8. The catheter of claim 7 further comprising biocompatible skull screws pre-attached and securely but freely spinning in the eyelets of the associated annulus set.

9. A peel-away electrode placement catheter comprising:
a silicone tube, the silicone tube including perforations longitudinally positioned to spilt the silicone tube in half;
a pair of separate metal semi-cylinders positioned within the silicone tube; and
a deep brain stimulation (DBS) implantable electrode positioned between the pair of separate metal semi-cylinders.

10. The peel-away electrode placement catheter of claim 9 further comprising:
a DBS lead linked to the electrode; and
a connector piece linked to the lead.

11. The peel-away electrode placement catheter of claim 9 wherein the silicone tube comprises:
a thickness of 0.1 millimeters;
a length of 21 centimeters; and
an overall diameter of 1.8 millimeters.

12. The peel-away electrode placement catheter of claim 9 wherein each of the separate metal semi-cylinders comprises:
a length of 19 centimeter; and
a radius of 0.3 millimeters.
